(19) United States
(12) Patent Application Publication
(10) Pub. No.: US 2022/0003959 A1
(43) Pub. Date: Jan. 6, 2022

(54) APPARATUS, METHOD, AND COMPUTER-READABLE MEDIUM FOR SELECTING AND MANUFACTURING OPTICAL LENSES

(71) Applicant: Essilor International, Charenton-Le-Pont (FR)

(72) Inventor: Michael FILIPOVICH, Dallas, TX (US)

(21) Appl. No.: 17/292,541

(22) PCT Filed: Nov. 12, 2019
(86) PCT No.: PCT/EP2019/081031

§ 371 Date: May 10, 2021

(51) Int. Cl. G02B 7/02

(52) U.S. Cl. CPC G02B 7/021; G02B 7/022

(57) ABSTRACT

An apparatus, method, and computer-readable medium for selecting and manufacturing of an optical lens that include receiving, via a network from a client device, prescription information, performing an initial search for a first optical lens that is compatible with the prescription information, determining additional information that, when provided, is usable to update the initial search, transmitting a result of the initial search along with a request for the additional information, determining a possible modification to the first optical lens or a second optical lens that is both compatible with the prescription information and associated with the additional information, transmitting an updated result, receiving a selection of an optical lens from the updated result, and manufacturing the selected optical lens or transmitting an instruction to an external manufacturing apparatus to manufacture the selected optical lens.

This application is the U.S. national phase of International Application No. PCT/EP2019/081031 filed Nov. 12, 2019 which designated the U.S. and claims priority to EP Patent Application No. 18315036.6 filed Nov. 12, 2018, the entire contents of each of which are hereby incorporated by reference.

### BACKGROUND OF THE INVENTION

### Field of the Invention

Embodiments described herein relate to the field of optical lens selection and manufacturing.

### Description of the Related Art

In the optical lens industry, it is often difficult to determine which optical lens out of a plurality of optical lenses is compatible with particular prescription criteria. In the optical lens industry, there is also currently no methodology to determine an optical lens that is not only compatible with the prescription criteria, but also with non-lens specific information.

In view of the drawbacks in the optical lens industry, the present disclosure sets forth a more robust and efficient lens design and manufacturing process with improved accuracy. This is facilitated by communication and processing among a plurality of appartuses over one or more networks.

### SUMMARY OF THE INVENTION

Described is an apparatus, method, and computer-readable medium for selecting and manufacturing of an optical lens. The apparatus includes processing circuitry configured to receive, via a network from a client device, prescription information that includes one or more of distance, addition, sphere, cylinder, axis, and prism, perform an initial search for a first optical lens that, out of a plurality of optical lenses, is compatible with the prescription information received from the client device, determine additional information that, when provided, is usable to update the initial search, the additional information including both lens-specific data and non-lens-specific data, transmit, via the network to the client device, a result of the initial search along with a request for the additional information, in response to receiving the additional information via the network from the client device, determine a possible modification to the first optical lens or a second optical lens that is both compatible with the prescription information and associated with the additional information, by evaluating previous selections of lenses performed at the client device or at other client devices at different locations, transmit, via the network to the client device, an updated result that includes the first optical lens as well as the possible modification to the first optical lens or the second optical lens, receive, via the network from the client device, a selection of an optical lens from the updated result out of one of the first optical lens, the first optical lens with the possible modification, and the second optical lens, and manufacture the selected optical lens or transmit an instruction to an external manufacturing apparatus to manufacture the selected optical lens.

This summary of the invention does not list all characteristics, and therefore, sub-combinations of these characteristics may also constitute an invention.

### BRIEF DESCRIPTION OF THE DRAWINGS

The disclosure will be better understood from reading the description which follows and from examining the accompanying figures. These figures are provided solely as non-limiting examples of the embodiments. In the drawings:

FIG. 1 illustrates a system of apparatuses/devices communicating via one or more networks according to one or more embodiments;

FIGS. 2 and 3 illustrate flowcharts of one or more processes according to one or more embodiments;

FIG. 4 illustrates a user interface of an order entry screen according to one or more embodiments;

FIG. 5 illustrates a user interface of a results display screen according to one or more embodiments; and

FIG. 6 illustrates a computer system upon which embodiments of the present disclosure may be implemented.

### DESCRIPTION OF THE PREFERRED EMBODIMENTS

One embodiment is drawn to an apparatus for selecting and manufacturing of an optical lens, the apparatus comprising: processing circuitry configured to receive, via a network from a client device, prescription information that includes one or more of distance, addition, sphere, cylinder, axis, and prism, perform an initial search for a first optical lens that, out of a plurality of optical lenses, is compatible with the prescription information received from the client device, determine additional information that, when provided, is usable to update the initial search, the additional information including both lens-specific data and non-lens-specific data, transmit, via the network to the client device, a result of the initial search along with a request for the additional information, in response to receiving the additional information via the network from the client device, determine a possible modification to the first optical lens or a second optical lens that is both compatible with the prescription information and associated with the additional information, by evaluating previous selections of lenses performed at the client device or at other client devices at different locations, transmit, via the network to the client device, an updated result that includes the first optical lens as well as the possible modification to the first optical lens or the second optical lens, receive, via the network from the client device, a selection of an optical lens from the updated result out of one of the first optical lens, the first optical lens with the possible modification, and the second optical lens, and manufacture the selected optical lens or transmit an instruction to an external manufacturing apparatus to manufacture the selected optical lens.

In one embodiment, the lens-specific data includes one of pantoscopic tilt and a corridor length, and the non-lens-specific data includes one of age, a medical condition, and a geographical location of a user who is associated with the prescription information.

In one embodiment, the processing circuitry is further configured to determine a timeline for manufacturing the selected optical lens based on whether materials and mechanisms used to manufacture the selected optical lens are available.

In one embodiment, the processing circuitry is further configured to determine, out of a plurality of possible external manufacturing apparatuses, the external manufacturing apparatus that is to manufacture the selected optical lens based on workload of each of the plurality of possible external manufacturing apparatuses.

In one embodiment, the processing circuitry is further configured to determine the possible modification to the first optical lens or the second optical lens that is both compatible with the prescription information and associated with the additional information, based on a trend among a particular age group a user belongs to, a hobby of the user, and a current time of the year.

One embodiment is drawn to a method performed by processing circuitry of an apparatus, the method comprising: receiving, by the processing circuitry via a network from a client device, prescription information that includes one or more of distance, addition, sphere, cylinder, axis, and prism; performing, by the processing circuitry, an initial search for a first optical lens that, out of a plurality of optical lenses, is compatible with the prescription information received from the client device; determining, by the processing circuitry, additional information that, when provided, is usable to update the initial search, the additional information including one of lens-specific data and non-lens-specific data; transmitting, by the processing circuitry via the network to the client device, a result of the initial search along with a request for the additional information; in response to receiving the additional information via the network from the client device, determining, by the processing circuitry, a possible modification to the first optical lens or a second optical lens that is both compatible with the prescription information and associated with the additional information, by evaluating previous selections of lenses performed at the client device or at other client devices at different locations; transmitting, by the processing circuitry via the network to the client device, an updated result that includes the first optical lens as well as the possible modification to the first optical lens or the second optical lens; and receiving, by the processing circuitry via the network from the client device, a selection of an optical lens from the updated result out of one of the first optical lens, the first optical lens with the possible modification, and the second optical lens.

In one embodiment, the lens-specific data includes one of pantoscopic tilt and a corridor length, and the non-lens-specific data includes one of age, a medical condition, and a geographical location of a user who is associated with the prescription information.

In one embodiment, the method further comprises determining, by the processing circuitry, a timeline for manufacturing the selected optical lens based on whether materials and mechanisms used to manufacture the selected optical lens are available.

In one embodiment, the method further comprises determining, by the processing circuitry, out of a plurality of possible apparatuses, an apparatus that is to manufacture the selected optical lens based on workload of each of the plurality of possible apparatuses.

In one embodiment, the determining of the possible modification to the first optical lens or the second optical lens that is both compatible with the prescription information and associated with the additional information, is based on a trend among a particular age group a user belongs to, a hobby of the user, and a current time of the year.

One embodiment is drawn to a non-transitory computer-readable storage medium including computer executable instructions, wherein the instructions, when executed by a computer, cause the computer to perform a method, the method comprising: receiving, via a network from a client device, prescription information that includes one or more of distance, addition, sphere, cylinder, axis, and prism; performing an initial search for a first optical lens that, out of a plurality of optical lenses, is compatible with the prescription information received from the client device; determining additional information that, when provided, is usable to update the initial search, the additional information including one of lens-specific data and non-lens-specific data; transmitting, via the network to the client device, a result of the initial search along with a request for the additional information; in response to receiving the additional information via the network from the client device, determining a possible modification to the first optical lens or a second optical lens that is both compatible with the prescription information and associated with the additional information, by evaluating previous selections of lenses performed at the client device or at other client devices at different locations; transmitting, via the network to the client device, an updated result that includes the first optical lens as well as the possible modification to the first optical lens or the second optical lens; and receiving, via the network from the client device, a selection of an optical lens from the updated result out of one of the first optical lens, the first optical lens with the possible modification, and the second optical lens.

In one embodiment, the lens-specific data includes one of pantoscopic tilt and a corridor length, and the non-lens-specific data includes one of age, a medical condition, and a geographical location of a user who is associated with the prescription information.

In one embodiment, the non-transitory computer-readable storage medium further comprises determining a timeline for manufacturing the selected optical lens based on whether materials and mechanisms used to manufacture the selected optical lens are available.

In one embodiment, the non-transitory computer-readable storage medium further comprises determining out of a plurality of possible apparatuses, an apparatus that is to manufacture the selected optical lens based on workload of each of the plurality of possible apparatuses.

In one embodiment, the determining of the possible modification to the first optical lens or the second optical lens that is both compatible with the prescription information and associated with the additional information, is based on a trend among a particular age group a user belongs to, a hobby of the user, and a current time of the year.

To brifely summarize, the present disclosure sets forth a more robust and more efficient optical lens design, selection and manufacturing process with improved accuracy. This is facilitated by communication and processing among a plurality of appartuses over one or more networks.

The capture of an eyewear (optical lens) order can be complicated. Typically an eyewear order is keyed into an order entry screen by an operator based on a fax, a hand written document that has been filled out by an eye care professional (ECP), or from data captured in an eyewear practice management system (PMS). It is common that the source of the information that is being used to enter the order is missing key pieces of data that are relevant or necessary to achieve a complete, accurate, and satisfactory representation of the desired optical lens to be manufactured or sourced on behalf of a patient who has requested the eyewear.

Furthermore, advancements in the optical industry have led to a plethora of new digitally produced optical lens designs that often require special parameters that were not necessary just a few years ago and which vary from product to product. For example, some lens products require the laterality or handedness of the patient (i.e., is the patient left-handed, right-handed, or ambidextrous). Certain lens products may require a measurement that is described as Near Vision Behavior, but not all lens products require this measurement. Thus, it is becoming more and more difficult for an order entry operator or patient to successfully enter a complete and accurate order that can be produced effectively.

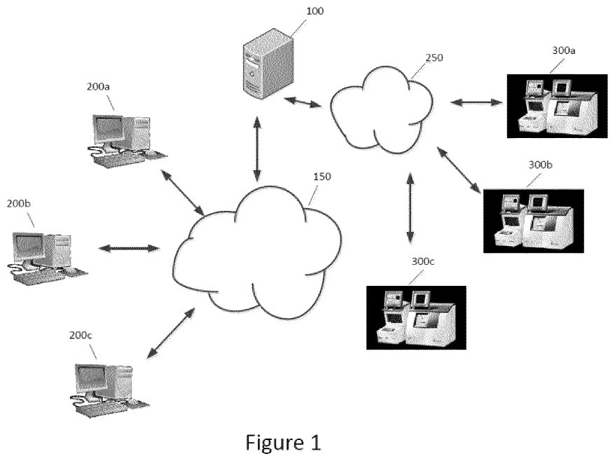

FIG. 1 shows an apparatus 100, various client devices (or client terminals) 200a, 200b, and 200c, and various lens manufacturing apparatuses 300a, 300b, 300c. Note that, unless a distinction needs to be made, the client devices 200a-200c may be used interchangeably to denote any or all of the devices and may be designated as “client device 200.” Similarly, unless a distinction needs to be made, the lens manufacturing apparatuses 300a-300c may be used interchangeably to denote any or all of the apparatuses and may be designated as “manufacturing apparatus 300.” Note that, the apparatus 100 may also include a manufacturing apparatus 300. In other words, the apparatus 100 and the manufacturing apparatus 300 may be one and the same device.

It is to be understood that there may be a plurality of (or only one of) each of these aforementioned components. Each of the client devices 200a-200c may communicate with apparatus 100 via one or more networks 150 (for example, a local area network (LAN) and/or another communications network, such as the Internet). Similarly, each of the manufacturing apparatuses 300a-300c may communication with apparatus 100 via one or more networks 250. Note that all of the above apparatuses/devices may also communicate amongst each other.

Note that, in one embodiment, the apparatus 100 may be part of one or more of the client devices 200a-200c. In other words, functions of the apparatus 100 may be performed by one or more of the client devices 200a-200c and vice versa.

Note that, in one embodiment, each of the apparatus 100, the client devices 200a-200c, and the manufacturing apparatuses 300a-300c may be in different geographical regions/areas (for example, in different cities, states, countries, etc.). Note that client device 200 may be located at a laboratory, a retail or commercial entity, an optician's office, or at a patient's home. Note that the client device 200 may be a mobile device. In one embodiment, the apparatus 100, the client devices 200a-200c, and the manufacturing apparatuses 300a-300c may all be on different networks, and it is not necessary for any apparatus/device to be on the same network as any other apparatus/device.

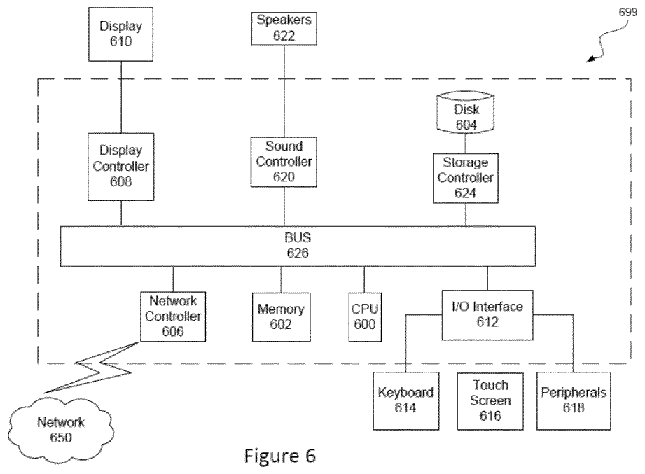

Each of the above-discussed apparatus 100 and client devices 200a-200c may be a server, computer (personal computer (PC), laptop, netbook, etc.), or the like which may operate with any operating system software. Each of the manufacturing apparatuses 300-300c may be any type of machine capable of making or producing optical lenses. For example, the machines may include a surface blocker, surface generator, engraver, edger, coating system, inspection device, or any other production device. Apparatus 100, client devices 200a-200c, and manufacturing apparatuses 300a-300c may each include the configuration shown in FIG. 6.

As described below in detail, the apparatus 100 is responsible for receiving data (for example, from the client device 200), processing data, and outputting data (for example, to the client device 200). Meanwhile, the client device 200 displays order entry screen(s) to assist a person (a patient or someone else) in placing an order for optical lenses/eyewear. The apparatus 100 controls what is being displayed via the order entry screen(s) based on the data being received, processing being performed, and data being output.

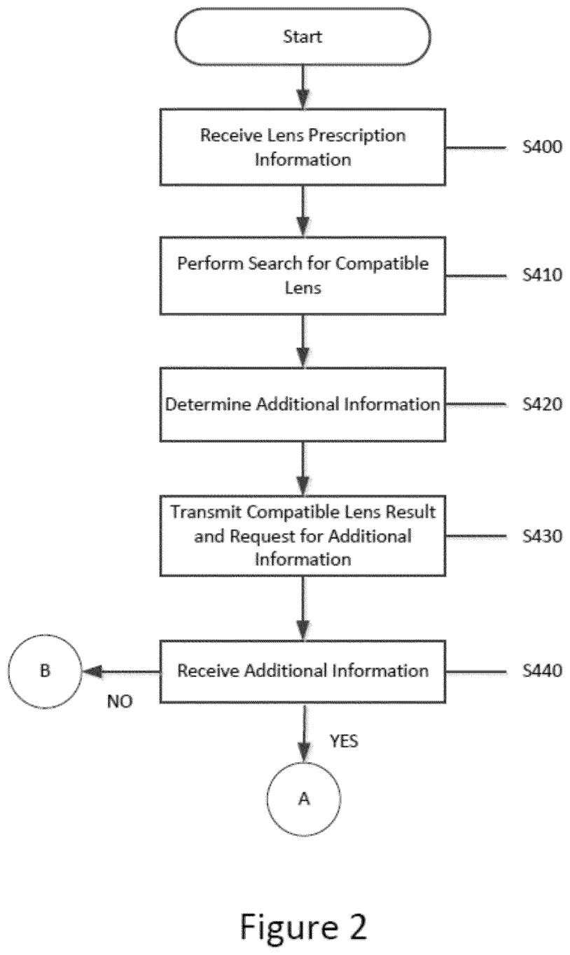

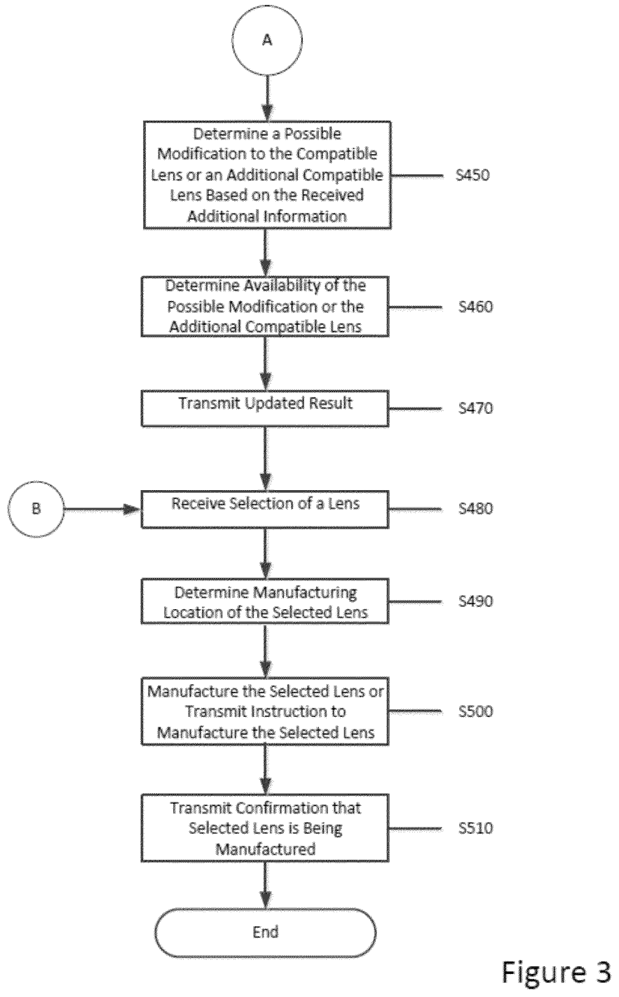

In one embodiment, in order for the apparatus 100 to perform the functions described below, in particular with reference to FIGS. 2 and 3, the apparatus 100 may receive lens data, for example via a network. Alternatively, the apparatus may employ a big data warehouse structure to obtain lens data. The lens data may relate to a wide range of parameters associated with optical lenses, contact lenses, eyewear, and the optical order process. Such parameters may include Sphere Power, Cylinder Power, Cylinder Axis, Prism amount, Prism Axis, Lens Material, Lens Style, Progressive or aspheric design, Lens base curve, Scratch Coating, Anti Reflective Coating, photochromic (for example, Transitions® products), Polarized products, etc.

Furthermore, apparatus 100 may also accumulate and analyze other information that is within the context of or related to optical lens/eyewear orders. Such information may include, for example, patient's hobbies/lifestyle (for example, active, sport, office, etc.), patient's gender, patient's age or age group, patient's address, point of purchase (retail vs. independent vs. consumer on-line, etc.), date of purchase, prefered date to receive lens, etc.

These aforementioned data may be accumulated in different ways either by the apparatus 100 or other device(s). Such data may be captured from previous orders, accumulated through aggregation from a group of cooperative devices, or accumulated through data feeds from a retail group as it captures orders or accumulated through an on-line ordering service. In one embodiment, the lens data may be imported to one or more data warehouses (which may be included within the apparatus 100 or external to the apparatus 100) in a pre-determined Extensible Markup Language (XML) format via a web-service, or in any of several file formats and transferred via processes or protocols such as File Transfer Protocol (FTP), Hypertext Transfer Protocol (HTTP), or other protocols.

The apparatus 100 or other device(s) may receive the lens data and then validate and store the lens data locally or remotely. The apparatus 100 or other device(s) may cleanse the lens data in different ways to eliminate duplicates, eliminate records with lens data that are outside of predetermined ranges, and store the lens data in one or more storage locations which may be cloud-based or internal to the entity that accumulates the lens data (for example, the apparatus 100). The lens data sets may be stored locally, regionally, or globally, and different lens data sets may be stored or aggregated in multiple forms and in multiple locations. The lens data sets may be stored in a fixed database or may be represented by one or more services that receives data in real-time from one or more services or a combination thereof. Again, this may be at the apparatus 100 side.

Apparatus 100 or other device(s) may apply an analytical engine that queries lens data from the data warehouse and/or from services that can feed the lens data that needed to be analyzed. The queries and analytics may be performed in real-time with direct access to the service from the order entry device, or at predetermined times or as a result of triggered events that can search for patterns or aggregate the lens data in ways that can demonstrate suggestive or qualitative ranges or listings that can be retrieved later.

The apparatus 100 or other device(s) may select lens data from its sources and perform a series of analyses on the lens data (described in further detail below, in particular, with respect to FIGS. 2 and 3). For example, the apparatus 100 or other device(s) may scan through large volumes of lens data and aggregate the lens data in smaller representations that may represent, for example, trends by time or by order parameter, groupings by lifestyle, gender, or address/location, statistical analysis including standard deviations about the mean, ranking analysis, analysis of variance, etc.

As an example, to demonstrate the use of the aforementioned lens data/analytics, consider a member in the cohort of a male population that is between 20 and 25 years of age with an active lifestyle. The highest ranked lens material in frequency of purchase selected by members of this cohort might be polycarbonate with an anti-reflective coating. Most likely this is because of polycarbonate material's tensile strength and protective qualities of both the lens material and the coating. However, the top ranked material in frequency of purchase for the cohort of females between 50 and 55 years of age with an active lifestyle might be a Transitions material with Transitions Brown color. Most likely this is because of the practicality of the Transitions® brand and ability for it to adapt automatically to indoor or outdoor light.

The aforementioned are examples of the type of lens data used by the apparatus 100 or other device(s) in one or more embodiments. The apparatus 100 or other device(s) may generate, via the analytical engine, these representations on a timed basis (e.g., every hour, every 24 hours, weekly, etc.) or may generate a representation ad-hoc based on a structured set of query parameters that may occur in real-time. In summary, in one embodiment, a purpose of the apparatus 100 or other device(s) may be to use the processing power of large calculation systems to break the lens data down into smaller and pre-calculated lens data sets that have relevance to the order entry systems that will eventually query it. This may include bell charts of data to show means, medians, standard deviations, etc. It may also show available choices or trends, etc. By analyzing the large lens data sets and representing them as smaller pre-analyzed sets, the smaller lens data sets will then be available quickly to a requesting service (such as the client device 200) that will need the lens data in real-time.

Again, this may be performed by the apparatus 100 itself or some other device that the apparatus 100 is in communication with. This structure provides, in effect, predictive selection such that data can be provided more quickly to an end user.

The analytical engine employed by the apparatus 100 or other device(s) may also include a suite of tools including fixed data sets, rules, and parameters that are aligned with product (i.e., lens) offerings. This suite may be used to associate fixed or optional parameter choices including default values that apply to the optical lens product and/or the order itself. For example, one optical lens product that can be selected from a catalog may require entry of the pantoscopic tilt (tilt of the lens bottom toward the patient's cheeks) for the patient's frame where another optical lens product may allow its entry as an option and yet another optical lens product may not require or even use pantoscopic tilt measurement if it's provided.

Another optical lens product may have a fixed corridor length (distance from the fitting point of the lens to some point along the corridor producing a minimum percentage of Add power (e.g., 85%)) choices available where a different optical lens product may allow a range of variable corridor lengths and yet another optical lens product may not even have a corridor. Thus, in one embodiment, the apparatus 100 may associate the need for the parameter (for example, corridor length) with the optical lens itself.

On the client device 200 side, an order entry system may integrate a tool that can form a request, submit that request to the apparatus 100, receive the results of that request, and display the data in a meaningful way to an order entry operator (or patient). The request may be a pre-defined XML data set that is delivered to the analytical engine of the apparatus 100 via a web-service, but the file format and delivery mechanism may vary according to the systems with which the analystical engine of the apparatus 100 interacts. Similarly the results may be delivered from the apparatus 100 to the requesting client device 200 by using different methods from system to system.

The content of the request may include instructions to the apparatus 100 as to the types of data that it needs. A wide variety of results choices may be available and these choices may be tailored to the request with various parameters within the XML data set request. The resulting data set may also include special offers, marketing information, or links, help documents or links to help documents, or other useful content that may be sponsored by a third party (such as a supplier or trusted vendor).

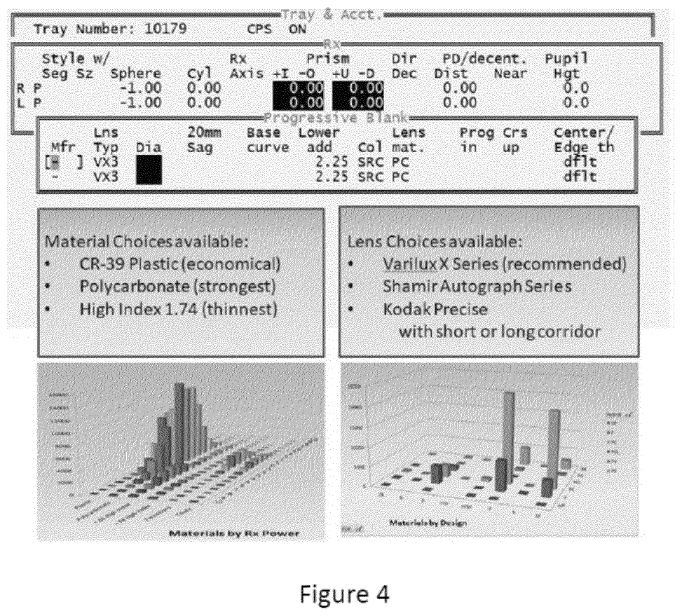

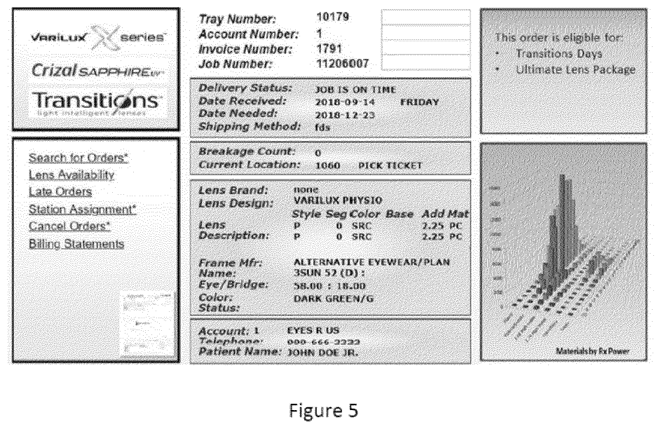

The formation of the request may be a result of a keyed order, a voice captured answer to a question or series of questions or a variety of other methods of data capture. Similarly the results may be displayed in charts, graphics, hyper-links, computer generated voice response, or other formats. FIGS. 4 and 5 are discussed below and respectively illustrate a user interface of an order entry screen and a user interface of a results display.

Since the tool on the client device 200 side is interactive, it may allow an operator or patient to drill-down into the results of a request and to submit additional requests that are alternatives to the original request or more specific than the first request. It may also feed the data from the request to the order entry system itself to allow the order entry system to default or to highlight the choices that it has received. For example, the initial order request may specify a lens material of CR-39 material with a nominal 6 base front curve. However, if the data and displays available to the order entry operator or patient suggest a lens material with higher index of refraction than the CR-39 material, then a 4 base front curve may be suggested by the apparatus 100 as a better choice than the 6 base. This may be due to the data aggregated/analytics which may indicate that a 4 base lens curve is more desireable as it is flatter and thus more fashionable than the 6 base lens.

In summary, the apparatus 100 is able to provide different type of suggestions (optical lens suggestions, material suggestions, coating suggestions, or the like), or recommendations based on the amount of information provided from the client device 200. Note that the aforementioned terms may be used interchangeably, and it is to be understood that “lens suggestions” may include suggestions of lenses, materials, coatings, etc.).

For example, with just the prescription information, the apparatus 100 is able to provide one or more compatible optical lens suggestions. However, once more information is provided to the apparatus 100, the apparatus 100 is able to provide even more lens suggestions which are a better fit for the patient and which may not have otherwise been suggested without the additional information.

The client device 200 is used to submit an instruction that leads to an order and manufacture of a pair of optical lenses (in one embodiment, along with eyeglasses or spectacles). This may be initiated via input by the person who the order is for (i.e., the patient) or by an employee (operator) at the particular location of the client device 200. The order entry at the laboratory or commercial entity can sometimes involve a conversation with an ordering optician.

During the ordering process, client device 200 displays order entry screen(s) to facilitate with the ordering process. Via the screen(s) and controlled by the apparatus 100, the client device 200 may ultimately display an order summary showing information of a price, a minimum thickness, a minimum lens blank diameter, etc. of the one or more lenses suggested by the apparatus 100, as further discussed below with respect to FIGS. 2 and 3.

In one embodiment, the order entry screen(s) may also display the most commonly selected lens material, coating, or other parameters that align with the patient's prescription. The order entry screen(s) may also display information regarding commonality, trends, or whether the given prescription might be exceptional for the chosen lens material, for example. FIGS. 4 and 5 below illustrate such example(s).

The processes summarized above are detailed below. First, a patient's lens prescription information is input at the client device 200. The prescription information may include some or all of the following: 1) distance vision (DV) or DISTANCE, which specifies the part of the prescription designed primarily to improve far vision; 2) near vision (NV), which may represent a single-vision lens prescription to improve near work, or the reading portion of a bifocal lens; 3) oculus dexter (OD), which is Latin for the right eye from the patient's point of view; 4) oculus sinister (OS), which is Latin for the left eye from the patient's point of view; 5) oculi uterque (OU), which is Latin for both eyes; 6) SPHERE, CYLINDER, and AXIS are values for describing the power of the lens using plus cylinder or minus cylinder notation; 7) ADD is an abbreviation for Near Addition, which is the additional refractive power to be combined, or added, to the distance power to achieve the ideal near power; 8) Prism and Base Prism, which refers to a displacement of the image through the lens, often used to treat strabismus and other binocular vision disorders; 9) Pupillary Distance (PD) or Interpupillary Distance (IPD), which is the distance between pupil centers; and 10) Back vertex distance (BVD), which is the distance between the back of the spectacle lens and the front of the cornea (the front surface of the eye). For example, the prescription information may include the information illustrated in Table 1, which is typically included in an eyeglass prescription:

TABLE 1

| | | SPHERE | CYLINDER | AXIS | PRISM | BASE |
|---|---|---|---|---|---|---|
| DISTANCE | OD | −4.00 | | | 0.5 | Down |
| | OS | −5.00 | −0.50 | 180 | 0.5 | Up |
| ADD | OD | +2.00 | | | | |
| | OS | +2.00 | | | | |

Note that in one embodiment, only the minimum amount of prescription information needed to manufacture an order may be provided from the client device 200 to the apparatus 100. In such embodiment, only the sphere power may be provided as this is sufficient prescription information in some cases to allow manufacture of an order.

According to FIGS. 2 and 3, the apparatus 100 receives, at S400, the patient's lens prescription information and determines at least one first lens, at S410, based on the prescription information. For example, the apparatus 100 may perform a search for one or more lens that would be compatible with the particular prescription. The apparatus 100 determines, for example, particular parameters associated with the prescription information. Such parameters may include, for example, lens material, lens coating, or other parameters that align with the given prescription. If the prescription is for a certain power, only one or two lens materials may be available. Similarly, only a specific type of lens coating may be available for the patient's particular prescription information.

Once the aforementioned determination is made, the apparatus 100 may also make a preliminary determination, for example based at least on the determined first optical lens and lens data, at S420, of other additional information that if provided by the patient, via the client device 200, could result in more specific (or customized) results. For example, as noted above, some lenses may require pantoscopic tilt, and if such information is not provided by the client device 200, such lens would not be part of the results. However, the apparatus 100 is able to make the determination that if additional information is provided (such as pantoscopic tilt), other lenses, materials, coatings, or the like can be provided in the search results as a recommendation to the patient.

Furthermore, aside from technical, lens-specific data/information such as pantoscopic tilt, corridor length, or the like (i.e., data that represents/is associated with the physical characteristics of the lens), the apparatus 100 may make a determination that other non-lens-specific data/information may be provided that would help give better results/suggestions to the patient (i.e., data that represents/is associated with characteristics of the patient). This information may include patient-specific information that concerns a wide-array of content. For example, patient-specific information may include patient name, address/location, age, insurance information, hobbies, lifestyle (active, inactive/sedentary, etc.), medical condition, whether the patient prefers eyeglasses or contact lenses, profession/working environment (in an office, outside, etc.), time spent in front of a computer monitor, length of time patient has been wearing eyeglasses or contact lenses, date the lenses are needed by (time constraints), or the like.

The patient-specific information may be useful in providing more customized search results to the particular patient. For example, if the patient leads an active lifestyle, apparatus 100 may make the determination to only present lenses to the patient that are lightweight. Similarly, if the patient is a teenager who plays soccer, for example, the apparatus 100 may make the determination to only present lenses (or special coatings/materials) with higher durability as this particular patient is at a high risk of having a normal (typically-recommended) lens break or scratch.

Further, business aspects of the transaction can affect the recommendations provided by the apparatus 100. With regard to insurance information, a specific insurance plan may have limited offerings or a large corporation may have a contract with the eye care provider (ECP) to provide a range of safety eyewear choices that fit the corporation's safety requirements. Thus, when this type of patient-specific information is provided to the apparatus 100, the apparatus 100 may use such information to provide more customized search results to the particular patient.

Based on the patient-specific information provided, the apparatus 100 may also offer health guidelines or advice. For example, California Proposition 65 warns of risks of birth defects or other risks that can be associated with purchase of the eyewear depending upon the lens material and/or the frame material. Thus, if the patient provides information that she is pregnant or expects to be (and identifies that she resides in California), the apparatus 100 may take this into account and not suggest certain lens and/or frame materials (and/or provide a warning of such materials).

Further to this concept, the apparatus 100 may also provide a warning that there may be a risk to a patient who does not choose an anti-reflective coating or ultraviolent (UV) blocker to protect against UV rays.

Upon receiving the initial information regarding the patient's prescription, the apparatus 100 is able to provide initial search results based on this limited information and also provide additional instructions or suggestions to enter more information (technical, lens-specific information such as pantoscopic tilt, corridor length, or the like, as well as non-lens-specific (patient-specific) information such as age, lifestyle, or the like) in order to refine the search and/or provide additional selections/modifications. As such, the apparatus 100 transmits, at S430, the initial/preliminary results to the client device 200 along with additional suggestions/inquiries.

Note that, in one embodiment, the apparatus 100 analyzes the particulars of the patient's prescription information and transmits, at S430, only the lens and/or lens parameters that are available for that prescription. In one embodiment, the apparatus 100 may also transmit, at S430, other parameters that do not necessarily align with the prescription information at the present time, but may for example be available in the near future. For example, if a particular type of coating will be available in a few days, weeks, or months, the apparatus 100 may also provide this type of information to the client device 200 in order to inform the patient. Current or future marketing or promotional offers may also be provided by the apparatus 100. For example, an offer to buy a second pair at 50% off might be available now or in the coming days. An offer may be available for specific products, so this could influence the decision of the patient to choose one product over another.

Subsequently, the client device 200 displays the transmitted results via the order entry screen(s) along with the additional suggestions/inquiries. For example, the results (suggested lenses/materials based on the preliminary prescription information) may be displayed in a list-type fashion. The suggestions/inquiries may be displayed in a question-like format: “Would you like to provide additional information, such as . . . ” or as a suggestion: “Please provide additional information, such as . . . in order to refine your results (or provide additional results).” Note that, in one embodiment, the apparatus 100 may actually provide suggestions/requests of the type of additional non-lens-specific (i.e., patient-specific) information needed to provide better (or additional) results. Thus, the apparatus 100 does not simply request “additional information,” but rather determines and/or requests what particular type of information should be provided (i.e., requests that the patient provide his/her age, address, hobbies, lifestyle, etc. information). This helps ensure better results and improves efficiency of the processing required on the apparatus 100 side, thereby achieved a technological advantage/effect.

Note that the patient may decide himself/herself what additional information, if any, to provide back to the apparatus 100. Thus, although the apparatus 100 may request/suggest additional information, the patient may choose not to provide any such additional information. In this case, the patient may simply select, via the client device 200 order entry screen(s), one of the lenses listed in the initial search results. Thus, the apparatus 100 would not receive any additional information from the client device 200 (NO at S440). At this point, the process would proceed to S480 in FIG. 3 in which the apparatus 100 would instead receive a selection of a lens from the compatible lens result transmitted at S430.

However, if the patient does decide to provide additional information, the client device 200 may receive, via input, this additional non-lens-specific (patient-specific) information about the patient. Again, this information may be provided at the request of the apparatus 100. The patient may decide to provide all requested information or only a portion of the requested information. In other words, if the apparatus 100 requested the age, address, and lifestyle of the patient may be provided, the patient may choose to only submit his/her age and address, but not the lifestyle.

In such case, the apparatus 100 receives (YES at S440) from the client device 200 the input information and uses this information to further refine the search results. As noted above, the refining of search results may include removing some of the previous lens suggestions and/or adding additional lens suggestions and/or adding additional modifications to the lenses initially suggested (for example, a special coat or polish) to the initial search results. Once the patient-specific information is received, the apparatus 100 performs a determination, at S450 in FIG. 3, of additional lenses that may suit this particular patient and/or other possible modifications to the one or more lenses already suggested at S430. In one embodiment, the apparatus 100 determines at least one of a possible modification to the first optical lens and a second optical lens based on the first determined lens and the additional information. Possible modifications may include, for example, different coatings, materials, or the like.

Assuming the apparatus 100 receives, as the patient-specific information, that the patient's age is “15” and the address is “Key West, Florida,” the apparatus 100 compares this information to information from other patients of the same or similar age. The apparatus 100 may first determine that patients of this age or similar age have ordered a special-type of light-weight lens. The apparatus 100 may also make additional predictions related to this age that may not necessarily be present in previously-gathered data. For example, the apparatus 100 may make an assumption that this patient may likely play sports, as 15-year olds are typically known to be active and involved in some type of sport. Based on this prediction, the apparatus 100 may make a determination that the special-type of light-weight lens may need a particular type of coating to be more scratch-proof.

Assuming the apparatus 100 receives, as the patient-specific information, hobbies that include scuba diving or boating, the apparatus 100 may determine that a certain type of lens (a lighter and thinner lens) is preferable over another type (a heavier and thicker lens) for this particular patient. Such determination may be made in view of other patients' lens selections in the past, patients who enjoy a similar hobby, lens specifications from the manufacturer, lens reviews (for example, reviews collected and analyzed from various sources including online customer reviews), etc. The apparatus 100 may also take into account fashion trends. Ratings or rankings from external sources (other devices that communicate with the apparatus 100 or stored at the apparatus 100 itself) that track fashion trends can be integrated to demonstrate or list offerings of color and style combinations for lenses and/or frames.

As another example, if the patient-specific information indicates that the patient has worn eyeglasses in the past and those eyeglasses have always been of the rimless-type, then apparatus 100 may determine that a certain type of lens is better equipped to be used in rimless eyeglasses. As a further example, if the patient-specific information indicates that the patient spends a considerable amount of time in front of a computer monitor, the apparatus 100 may generate a suggestion of a lens (or certain type of coating) that reduces glare.

The apparatus 100 also performs an analysis on ordering trends of other patients in “Key West, Florida” as well as any other location that may be similar—for example, other beach towns, places in warm climates, etc. With this analysis, the apparatus 100 may determine that a predominant number of patients in Key West, Florida (as well as other similar locations) have ordered photochromic lenses (a lens that darkens in the sun, as opposed to regular lenses—likely due to the sunny climate).

However, the apparatus 100 does not just make individual determinations based on each piece of information received from the client device 200. In such case, the apparatus 100 would simply provide results of the light-weight lens with the scratch-proof coating as well as photochromic lenses—thereby making in some cases the results too burdensome for the patient (i.e., too many choices). As such, the apparatus 100 goes a step further and performs an analysis on the all patient-specific information provided in combination. Thus, apparatus 100 does not simply look at “age” and “address” separately, but rather compares its findings for both in order to provide improved results to the patient.

Accordingly, the apparatus 100 analyses the information of age “15” and address “Key West, Florida” collectively. To this end, the apparatus 100 may determine that although patients in Key West, Florida usually order photochromic lenses, none of these patients are of a similar age of “15.” Thus, the apparatus 100 may determine that although by location photochromic lenses are popular in “Key West, Florida” or other similar locations, these lenses are not popular among 15-year olds. As such, the apparatus 100 may make the determination that photochromic lenses should not actually be suggested to this particular patient that is 15-years old. The apparatus 100 may also go a step further and compare the trends of 15-year olds among other regions or countries in order to determine whether photochromic lenses are not popular among 15-year olds in “Key West, Florida” or not popular among 15-year olds anywhere. This can help solidify the decision by the apparatus 100 not to suggest photochromic lenses to this particular patient that is 15-years of age. If, however, the apparatus 100 determines that 15-year olds outside of Key West, Florida do indeed order photochromic lenses, the apparatus 100 may actually suggest this type of lenses to the patient from Key West, Florida.

The apparatus 100 may also adapt its analysis, determinations, and suggestions based on its own success rate over a period of time. For example, if the apparatus 100 using mainly historic data suggests three choices of lens material, the apparatus 100 may also review the number of times the patient changed his/her choice from an original decision to one of the three suggested choices provided by the apparatus 100. Thus, the influence of the interactive process described herein may be used by the apparatus 100 to adjust the ranking toward products (lenses, materials, coatings, etc.) that are more likely to be selected as a result of the process described herein.

The apparatus 100 may also take into account sponsorship and provide suggestions in view of particular sponsor(s). For example, the suppliers of products can offer incentives to display given products via the apparatus 100 even though the products might not rank highly otherwise. This concept is a form of advertising to help promote products that might be too new or revolutionary to rank effectively based on historic or aggregated data sets. Thus, in following the above example of the patient that is 15-years of age, the apparatus 100 may suggest a particular type of lens to this patient even though historical data would not otherwise support such a suggestion. For example, although no 15-year old patient has ever selected lens A, the apparatus 100 may still suggest lens A to such a patient.

The apparatus 100 may also use medical knowledge when suggesting a particular lens. For example, the medical condition anisometropia exists when the two eyes of the patient differ in refractive power by two or more diopters. For patients that have four or more diopter difference in refractive power, the apparatus 100 may suggest a slab-off lens to address the prismatic effect at the near vision point. Thus, in following the above example of the patient that is 15-years of age, the apparatus 100 may suggest a slab-off lens even though trends or “what is popular among 15-year olds” may not suggest such a lens. Such a recommendation by the apparatus 100 would be as a result of this patient's particular medical condition, and may not be recommended to other patients within this group or geographical location that do not have such a condition. Note that such a medical condition may be included as part of the patient-specific information received from the client device 200.

Further, note that the apparatus 100 may rank “medical condition” information higher in its decision-making process than other information provided by the patient (i.e., treat it as being more important). Thus, if a particular medical condition would suggest a recommendation for lens A, but lens A is not popular among, for example, 15-year olds, the apparatus 100 would still recommend lens A as “medical condition” is ranked higher (given greater importance) than trends or what is popular within an age group.

The apparatus 100 may also perform additional analysis on the patient-specific information received from the client device 200. For example, the apparatus 100 may determine what is most important in a lens to patients of that age group. These data can be used not only from regional patients, but from patients from other regions as well. For example, it may be determined that to patients of 15-years of age, style is most important and trumps all other criteria such as comfort, shape, etc. Accordingly, in such case, the apparatus 100 may only present the lenses that are in-style or trendy among patients of 15-years of age or among patients of a particular age group (for example, 12-16 years of age). Thus, different ages or age groups may receive different lens recommendations. Similarly, different locations (city, state, zip code, etc.) may receive different lens recommendations. As an example, someone living in Alaska may not receive a recommendation for a photochromic lens as someone living in Florida might even if similar patient-specific information is provided for both patients (i.e., same age, same lifestyle, etc.). Similarly, someone living in a major city may receive a different lens recommendation than someone living in a more rural area.

Similarly, the apparatus 100 may provide different recommendations based on the season. For example, as the season turns toward summer, the apparatus 100 may recommend sun-wear choices more prominently, whereas moving into the winter season in northern climates, the apparatus 100 may recommend an anti-fog coating to help prevent the lenses from fogging over when coming in from the cold outdoors.

Note that the apparatus 100 may not actually receive as the patient-specific information details on the particular season. However, the apparatus 100 may determine the season based on the particular date and assume that as the winter season is approaching, the anti-fog coating should be recommended. In other words, the apparatus may use information not specifically indicated by the patient from client device 200 in order to make its determination.

Assuming that the apparatus 100 receives, as the patient-specific information, the patient's age as “15,” the address as “Key West, Florida,” and also the patient's hobby as “video games,” then, aside from the analysis performed above, the apparatus 100 may no longer make the assumption that this patient may likely play sports, as 15-year olds are typically known to be active and involved in some type of sport. Rather, this assumption would be replaced by the hobby of “video games”—which the apparatus 100 may determine that a lens with a special type of coating to reduce glaring may be a better suggestion for this particular patient who enjoys video games.

Additionally, the apparatus 100 may also perform an analysis by comparing other patients (in the same city, state, region, country, continent, etc.) that have a similar video game hobby and that are 15-years of age or within a particular age range. The apparatus 100 may determine, for example, that such individuals usually do not play as many (or any) sports as individuals of the same age/age group. As such, for this particular patient, the apparatus 100 may determine that a lens that reduces glare is a better fit than a lens that is more scratch-proof. Of course, if the patient indicates hobbies as both sports and video games, the apparatus 100 would again render a different result—a lens that reduces glare as well as is scratch resistant. In other words, any type of patient-specific information submitted to the apparatus 100 may cause the apparatus 100 to return a different result (i.e., a different lens/material suggestion).

Similarly, the more information that is provided by the patient, the better of a determination the apparatus 100 is able to make. As noted above, if no information on hobbies was submitted by the patient, then the apparatus 100 may make the assumption that the patient plays sports and thus would have recommended a lens that may not have been as suitable for a patient of the same age/age group that prefers video games. Thus, when minimal patient-specific information is provided from the client device 200 to the apparatus 100, the apparatus 100 “fills in the gaps” by making certain assumptions about the patient based on the patient-specific information that is indeed provided. Note that when the results are finally presented to the patient on the order entry screens at the client device 200, one or all of the lenses (and/or lens parameters) listed may include an explanation as to how the apparatus 100 came to the determination to suggest that particular lens (and/or lens parameters).

In one embodiment, the apparatus 100 may (either at the request of the patient via the client device 200 or without any specific request) make a determination, at S460, as to the availability of each of the suggested lenses. For example, assuming that for the above-discussed example of a 15-year old from Key West, Florida, the apparatus 100 has determined two lenses—lens A and lens B, the apparatus 100 would next make a determination of the availability of each of these lenses to be manufactured. The manufacturing of a lens can be performed at the apparatus 100 itself, a local apparatus that is different from the apparatus 100 (for example, an apparatus located at the same location as the apparatus 100), or at a remote apparatus that is located external to the apparatus 100 (for example, in a different city, state, region, country, etc.).

Nonetheless, the apparatus 100 determines the timeline for manufacturing of lens A and lens B. This determination may be achieved by inquiring either within the apparatus 100 itself (if the apparatus 100 includes lens manufacturing capabilities) or some other apparatus, whether lenses are available to be produced to the required specifications, as well as whether the particular mechanisms that perform the cutting, coating, polishing, etc. are also functioning and able to produce the optical lens. The apparatus 100 may receive as a response, either internally or from another apparatus, that the particular lens can be manufactured right away. However, the apparatus 100 may receive a response that lens A can be manufactured right away, but lens B cannot be manufactured right away. For example, the mechanism within apparatus 100 (or in another apparatus) that produces the particular polish or tint, etc. of lens B may have malfunctioned or may be undergoing service. As a result, this information may be relayed to the apparatus 100 so that the apparatus 100 can use this information to make a better determination as to which lens (for example, lens A or lens B) to suggest to the patient or to simply inform the patient of the additional wait time to manufacture lens B.

The apparatus 100 may also determine which lenses (and/or lens parameters) to recommend to the patient based on currently-available inventory. For example, if there is an overabundance of photochromic lenses available either locally (to Key West, Florida) or remotely (some other city, state, region, or country), the apparatus 100 may increase its recommendation of photochromic lenses in an effort to not only reduce its inventory size but also to suggest something to patients who have never considered photochromic lenses. The apparatus 100 may also offer incentives to the patient for selecting a photochromic lens (for example, lower price, quicker manufacturing time, faster shipping).

Similarly, the apparatus 100 may also control the inventory of particular lenses (for example, photochromic lenses) in areas in which the apparatus 100 typically makes such recommendation. For example, as noted above, since the apparatus 100 may recommend photochromic lenses in areas of warm climate, the apparatus 100 may also control the increase of the inventory of photochromic lenses in those areas (or in the areas that service the warm client areas). For example, if a particular manufacturing center services Key West, Florida, the apparatus 100 may communicate with the manufacturing apparatus 300 at the manufacturing center and issue an instruction to increase inventory of photochromic lenses by, for example, 20%. This can be done by the apparatus 100 transmitting a message to the manufacturing apparatus 300, which in turn gets displayed to an operator at the corresponding manufacturing center. The apparatus 100 may also instruct one manufacturing center/apparatus 300 to send photochromic lenses to a manufacturing center/apparatus 300 in order to better distribute the lenses to areas where they are in higher demand (or areas in which the apparatus 100 recommends these lenses the most).

Next, the apparatus 100 transmits, at S470, to the client device 200 the updated list of recommended lenses (and/or lens parameters) for the patient based on the initial prescription information provided as well as the additional information (lens-specific information and/or patient-specific information), and controls the client device 200 to display the updated list. Note again that the updated list may include some or all of the lenses (and/or lens parameters) included in the original lens list (resulting from the prescription information), and/or other lenses (and/or lens parameters) not included in the original lens list, etc. In other words, the lenses in the original list may be completely replaced by other lenses resulting from the additional information provided.

Once the updated list of recommended lenses (and/or lens parameters) is displayed, the client device 200 awaits an input of a selection of one of these lenses (and/or lens parameters). Such selection may be input by the patient after he/she has studied the recommended lenses (and/or lens parameters) and decided to move forward or purchase a particular one of the lenses. This selection is input at the client device 200 via the order entry screens, and is then transmitted to the apparatus 100.

As a result, the apparatus 100 receives, at S480, the selection of the particular lens (and/or lens parameters) from the client device 200. Once this is received, the apparatus 100 may perform a determination, at S490, as to where to perform the manufacturing of the selected lens (for example, at the apparatus 100 itself, some other local apparatus, or some remote apparatus). At S490, the apparatus 100 determines the most efficient manufacturing center/apparatus 300 for the manufacturing of the particular selected lens, so that the lens can be delivered to the patient as quickly and as efficiently as possible.

This determination may be based on various criteria. For example, the apparatus 100 may make this determination based on workload of a particular manufacturing apparatus 300, based on inventory, based on turn-around time, etc. For example, the apparatus 100 may determine that the closest manufacturing center/apparatus 300a to the patient's location (for example, Key West, Florida) is backed-up with other orders, whereas a manufacturing center/apparatus 300b in another location (for example, Alexandria, Virginia) only has two other orders queued up.

The apparatus 100 may further determine that although the manufacturing apparatus 300b in Alexandria, Virginia is farther away, the lens can be manufactured much quicker. The apparatus 100 may also calculate the length of time it takes to ship the lens from Alexandria, Virginia to the patient's location (for example, Key West, Florida), to be two (2) days. As a result, the apparatus 100 may conclude that although Alexandria, Virginia is farther away from the closest manufacturing center/apparatus 300a to the patient's location, it will be more efficient (and quicker) for the lens to be manufactured in Alexandria, Virginia and shipped to the patient's location than it would be to manufacture it in the center closest to Key West, Florida.

Further, assuming the apparatus 100 determines that the closest manufacturing center/apparatus 300c that can manufacture the selected lens right away is in Los Angeles, California, the apparatus 100 would also calculate the length of time it would take to ship the lens from Los Angeles, California. Assuming the apparatus 100 determines that it would take four (4) days for such shipment from Los Angeles, Calif., the apparatus 100 may then determine that although the center/apparatus 300c in this location can manufacture the lens right away, it would still take a longer time to receive this lens (due to the shipment). Accordingly, the apparatus 100 may make the determination to instruct the manufacturing center/apparatus 300a closest to the patient's location to manufacture this lens, although this center/apparatus 300a may actually have many manufacturing orders already queued.

The apparatus 100 may also perform the aforementioned determinations by taking into account historical data of different manufacturing centers/apparatuses 300. For example, if the patient selects the lens in the month of August, the apparatus 100 may determine based on past data that the manufacturing centers in Alexandria, Virginia, Los Angeles, Calif., and closest to Key West, Fla. are usually very busy during this time, while centers in Cleveland, Ohio and Albany, New York are usually less busy during this time. Thus, the apparatus 100 may inquire only the centers/apparatuses 300 that are historically less busy in the month of August about current workload, as opposed to inquiring every center, for example, in the U.S. region. This helps streamline the process and provides the technological advantage/technical effect of minimizing processing of the apparatus 100 by only sending inquiries to manufacturing centers/apparatuses 300 that are historically less busy during the particular time at which the lens is to be manufactured.

Once the apparatus 100 has determined the manufacturing center/apparatus 300 that is to manufacture the particular selected lens, the apparatus 100 transmits an instruction, at S500, to the manufacturing center/apparatus 300 to manufacture the lens along with the specifications, parameters, etc. of the lens or manufactures the lens at the apparatus 100 (assuming the apparatus 100 is capable of manufacturing the lens). Next, the apparatus 100 receives a confirmation from the manufacturing apparatus 300 that the instruction was received and that the lens manufacturing process will commence. Along with the confirmation, the manufacturing apparatus 300 may also send an estimated time of completion of the manufacturing process. If the lens manufacturing process is to be carried out at the apparatus 100, then the apparatus 100 itself would generate such confirmation.

After the confirmation is received from the manufacturing apparatus 300, the apparatus 100 may send a confirmation, at S510, to the client device 200 indicating that the manufacturing process is underway and may also include an estimated time by which the patient should receive the manufactured lens.

Note that, with respect to S440 in FIG. 2, even if the patient decides not to provide any additional information, the apparatus 100 may still use limited information that it can acquire on the patient in order to perform the processes discussed above, in particular with reference to S450-S510. For example, although the patient may not provide an address, the apparatus 100 may make a determination of the location of the patient based on the location of the client device 200 with which the apparatus 100 communicates. If the client device 200 is, for example, located in an optician's office in Key West, Fla., the apparatus 100 may make an assumption that the patient's location is in Key West, Fla. or at least thereabout. The apparatus 100 may then use, similar to the above-discussed processes, big data of previous orders in the Key West, Fla. area or thereabout to determine suggestions for this particular patient based on trends, etc.

Thus, the apparatus 100 may interpolate data of previous orders in the Key West, Fla. area or thereabout to determine a possible modification to the compatible lens or to determine other compatible lenses for the patient as discussed above with reference to S450 of FIG. 3, even without the patient actually providing this data at S440. Of course, the apparatus 100 may use other information that it can acquire on the patient—for example, age, sex, etc. This can be achieved by the apparatus 100 analyzing past orders of this particular patient (assuming this person is not a first-time patient) or making a determination that the patient is male based on the name, for example, and then performing determinations as performed with reference to S450 based on, for example, what males of age 30 that live in the Key West, Fla. region have previously purchased. Thus, even if the patient does not wish to input additional information, the apparatus 100 can still perform meaningful determinations of lenses or lens parameters that would be compatible for this particular patient. This may help eliminate additional burden of the patient to input more information.

The apparatus 100 may include an exchange service that can interact with reference to S450 and/or S440 to allow an initial analysis to be made and submitted in real-time to S450 and/or S440 such that the ECP or the patient can respond to accept, reject, or in other ways modify its response and thus become an interactive system that helps drive the best result for that particular patient.

FIG. 4 illustrates a user interface of an order entry screen according to one embodiment. Note that the user interface of both FIGS. 4 and 5 are displayed at the client device 200 (and such display is controlled by the apparatus 100, as discussed above). In one embodiment, the apparatus 100 generates such interface(s) or controls generation of such interface(s). In this example, the patient or the ECP may have input the “Rx” (prescription information) and “Progressive Blank” information at the client device 200 (i.e., the information at the top of the figure). Once the processing is performed by the apparatus 100, the particular recommendations may be displayed (i.e., the information at the bottom of the figure). In this particular example, the apparatus 100 determined that the material choices available for this particular patient are: “CR-39 Plastic” (the economical choice), “Polycarbonate” (the strongest choice), and “High Index 1.74” (the thinnest choice). Similarly, the apparatus 100 determined that the lens choices available for this particular patient are “Varilux X Series,” “Shamir Autograph Series,” and “Kodak Precise” (with short or long corridor). Note that out of these “available” recommended choices, the apparatus 100 recommends “Varilux X Series” the most. This recommendation may be based on one or more of the factors discussed above with respect to FIGS. 2 and 3 (age, lens style, lifestyle, trends, historical orders, seasons, geographical location, medical condition, sponsorship, etc.). Furthermore, the user interface of FIG. 4 may also display various graphs/charts illustrating popular (or available) lens materials in view of prescription power and/or design. Note that additional graphs/charts may also be displayed that show popularity of one or more lenses by age, lens style, lifestyle, trends, historical orders, seasons, geographical location, medical condition, sponsorship, etc.

FIG. 5 illustrates a user interface of a results display screen according to one embodiment. In one embodiment, this user interface may be displayed after S510 of FIG. 3 is complete, at the end of the process discussed with reference to FIG. 3. In one embodiment, this user interface may be displayed at a later time (i.e., once the patient or ECP goes back into the system to check the status of an order).

FIG. 5 may display some information that is similar to the information displayed in FIG. 4. Further, the user interface of FIG. 5 may illustrate information such as delivery status, date order was received, date order is needed by, shipping method, breakage count, current location, etc. This user interface may also display the particular lens brand selected by the patient or ECP (if any), the lens design, lens description, frame manufacturer, color, account (patient's account or the account of ECP—i.e., the location where the order was placed), patient's phone number or contact information, as well as patient name

Note that in the example of FIG. 5, the user interface also displays additional information such as “this order is eligible for transitions days and ultimate lens package.” This information may also be recommended/suggested by the apparatus 100 based on the analysis discussed above with reference to FIGS. 2 and 3. In fact, this information may be linked to the “sponsorship” concept in that if a promotion is available or if the manufacturer is offering a special deal or package, the apparatus 100 may provide this information in addition to the recommendation of the particular lens or lens material.

Note that, in one embodiment, the user interface of FIG. 5 may display links to search for orders, lens availability, late orders, station assignment, cancel orders, billing statements, etc. Further, in one embodiment, graphs/charts may be displayed (which may be similar to the ones illustrated in FIG. 4). For example, these graphs/charts may illustrate popular (or available) lens materials in view of prescription power, etc.

Note that the user interfaces of FIGS. 4 and 5 provide technological improvements. With such user interfaces, the data are easily displayable to a user (such as a patient or a novice ECP) who may not be completely familiar with the terminology/details pertaining to optical lenses or materials. Without such user interfaces, a user would have to drill down through many pieces of data (or pages) to get to desired data/information, which would be slow, complex, and difficult to comprehend, particularly for novice users. The user interfaces of FIGS. 4 and 5 address this issue and provide the technical advantage of saving the user time both in navigating to the desired information and also in understanding the meaning of such information.

The method of FIGS. 2 and 3 as well as each of the functions of the above described embodiments may be implemented by circuitry, which includes one or more processing circuits. A processing circuit includes a particularly programmed processor, for example, processor (CPU) 600, as shown in FIG. 6. A processing circuit also includes devices such as an application specific integrated circuit (ASIC) and conventional circuit components arranged to perform the recited functions.

In FIG. 6, the device 699 includes a CPU 600 which performs the processes described above. The device 699 may be a general-purpose computer or a particular, special-purpose machine. In one embodiment, the device 699 becomes a particular, special-purpose machine when the processor 600 is programmed to perform one or more functions outlined in the present specification.

Note that device 699 may be a server, a personal computer (PC), a tablet, a cellular/smart phone. Hence, the embodiments discussed herein may be implemented on any of these devices. Furthermore, the device 699 may also include components for manufacturing optical lenses, or be part of a lens manufacturing device (for example, Mr. Blue 2.0, Mr. Orange, Neksia, Delta 2™, Pro-E 600, or the like).

The process data and instructions may be stored in at least one computer readable medium or memory 602 for holding the instructions programmed according to any of the teachings of the present disclosure and for containing data structures, tables, records, or other data described herein. These processes and instructions may also be stored on a storage medium disk 604 such as a hard drive (HDD) or portable storage medium or may be stored remotely. The instructions may be stored on CDs, DVDs, in FLASH memory, RAM, ROM, PROM, EPROM, EEPROM, hard disk or any other device with which the system communicates, such as a server or computer.

Further, the discussed embodiments may be provided as a utility application, background daemon, or component of an operating system, or combination thereof, executing in conjunction with CPU 600 and an operating system such as, but not limited to, Microsoft Windows, UNIX, Solaris, LINUX, Android, Apple MAC-OS, Apple iOS and other systems known to those skilled in the art.

CPU 600 may be any type of processor that would be recognized by one of ordinary skill in the art. For example, CPU 600 may be a Xenon or Core processor from Intel of America or an Opteron processor from AMD of America. CPU 600 may be a processor having ARM architecture or any other type of architecture. CPU 600 may be any processor found in a mobile device (for example, cellular/smart phones, tablets, personal digital assistants (PDAs), or the like). CPU 600 may also be any processor found in computer and/or specific lens manufacturing apparatuses.

Additionally or alternatively, the CPU 600 may be implemented on an FPGA, ASIC, PLD or using discrete logic circuits, as one of ordinary skill in the art would recognize. Further, CPU 600 may be implemented as multiple processors cooperatively working in parallel to perform the instructions of the processes described herein. The computer 699 in FIG. 6 also includes a network controller 606, such as, but not limited to, a network interface card, for interfacing with network 650 (which may be the same as or similar to networks 150 and 250 of FIG. 1). As can be appreciated, the network 650 can be a public network, such as, but not limited to, the Internet, or a private network such as an LAN or WAN network, or any combination thereof and can also include PSTN or ISDN sub-networks. The network 650 can also be wired, such as an Ethernet network, or can be wireless such as a cellular network including EDGE, 3G and 4G wireless cellular systems. The wireless network can also be WiFi, Bluetooth, or any other wireless form of communication that is known.

The computer 699 further includes a display controller 608, such as, but not limited to, a graphics adaptor for interfacing with display 610, such as, but not limited to, an LCD monitor. A general purpose I/O interface 612 interfaces with a keyboard and/or mouse 614 as well as a touch screen panel 616 on or separate from display 610. General purpose I/O interface also connects to a variety of peripherals 618 including printers and scanners. The peripheral elements discussed herein may be embodied by the peripherals 618 in the exemplary embodiments.

A sound controller 620 may also be provided in the computer 699 to interface with speakers/microphone 622 thereby providing sounds/audio. The speakers/microphone 622 can also be used to accept dictated words as commands

The general purpose storage controller 624 connects the storage medium disk 604 with communication bus 626, which may be an ISA, EISA, VESA, PCI, or similar. A description of the general features and functionality of the display 610, keyboard and/or mouse 614, as well as the display controller 608, storage controller 624, network controller 606, sound controller 620, and general purpose I/O interface 612 is omitted herein for brevity as these features are known.

Obviously, numerous modifications and variations of the present disclosure are possible in light of the above teachings. It is therefore to be understood that within the scope of the appended claims, the invention may be practiced otherwise than as specifically described herein.

For example, advantageous results may be achieved if the steps of the disclosed techniques were performed in a different sequence, if components in the disclosed systems were combined in a different manner, or if the components were replaced or supplemented by other components.

The functions, processes, and algorithms described herein may be performed in hardware or software executed by hardware, including computer processors and/or programmable processing circuitry configured to execute program code and/or computer instructions to execute the functions, processes, and algorithms described herein.

The functions and features described herein may also be executed by various distributed components of a system. For example, one or more processors may execute these system functions, wherein the processors are distributed across multiple components communicating in a network (such as networks 15, 250, 650, or any other network). The distributed components may include one or more client and/or server machines, in addition to various human interface and/or communication devices (e.g., display monitors, cellular/smart phones, tablets, PDAs). The network may be a private network, such as a LAN or WAN, or may be a public network, such as the Internet. Input to the system may be received via direct user input and/or received remotely either in real-time or as a batch process.

Additionally, some implementations may be performed on modules or hardware not identical to those described. Accordingly, other implementations are within the scope that may be claimed.

It should be noted that, as used in the specification and the appended claims, the singular forms “a,” “an,” and “the” include plural referents unless the context clearly dictates otherwise. Further, the embodiments discussed herein may be combined together in any fashion.

1. An apparatus for selecting and manufacturing of an optical lens, the apparatus comprising:
processing circuitry configured to
receive, via a network, lens data; <receive, via a network from a client device, prescription information that includes one or more of distance, addition, sphere, cylinder, axis, and prism,
determine a first optical lens based on the prescription information received from the client device,
determine additional information to request based at least on the determined first optical lens and lens data, the additional information including both lens-specific data and non-lens-specific data,
transmit, via the network to the client device, a result of the initial search along with a request for the additional information,
in response to receiving the additional information via the network from the client device, determine at least one of a possible modification to the first optical lens and a second optical lens based on the first determined optical lens and the additional information
transmit, via the network to the client device, an updated result that includes the first optical lens as well as the possible modification to the first optical lens or the second optical lens,
receive, via the network from the client device, a selection of an optical lens from the updated result out of one of the first optical lens, the first optical lens with the possible modification, and the second optical lens, and
manufacture the selected optical lens or transmit an instruction to an external manufacturing apparatus to manufacture the selected optical lens.

2. The apparatus according to claim 1, wherein the lens-specific data includes one of pantoscopic tilt and a corridor length, and the non-lens-specific data includes one of age, a medical condition, and a geographical location of a user who is associated with the prescription information.

3. The apparatus according to claim 1, wherein the processing circuitry is further configured to determine a timeline for manufacturing the selected optical lens based on whether materials and mechanisms used to manufacture the selected optical lens are available.

4. The apparatus according to claim 1, wherein the processing circuitry is further configured to determine, out of a plurality of possible external manufacturing apparatuses, the external manufacturing apparatus that is to manufacture the selected optical lens based on workload of each of the plurality of possible external manufacturing apparatuses.

5. The apparatus according to claim 1, wherein the processing circuitry is further configured to determine the possible modification to the first optical lens or the second optical lens that is both compatible with the prescription information and associated with the additional information, based on a trend among a particular age group a user belongs to, a hobby of the user, and a current time of the year.

6. A method performed by processing circuitry of an apparatus, the method comprising:
receiving, by the processing circuitry, lens data;
receiving, by the processing circuitry via a network from a client device, prescription information that includes one or more of distance, addition, sphere, cylinder, axis, and prism;
determining a first optical lens based on the prescription information received from the client device;
determining, by the processing circuitry, additional information to request based at least on the determined first optical lens and lens data, the additional information including one of lens-specific data and non-lens-specific data;
transmitting, by the processing circuitry via the network to the client device, a result of the initial search along with a request for the additional information;
in response to receiving the additional information via the network from the client device, determining, by the processing circuitry, at least one of a possible modification to the first optical lens and a second optical lens based on the first determined optical lens and the additional information
transmitting, by the processing circuitry via the network to the client device, an updated result that includes the first optical lens as well as the possible modification to the first optical lens or the second optical lens; and
receiving, by the processing circuitry via the network from the client device, a selection of an optical lens from the updated result out of one of the first optical lens, the first optical lens with the possible modification, and the second optical lens.

7. The method according to claim 6, wherein the lens-specific data includes one of pantoscopic tilt and a corridor length, and the non-lens-specific data includes one of age, a medical condition, and a geographical location of a user who is associated with the prescription information.

8. The method according to claim 6, further comprising determining, by the processing circuitry, a timeline for manufacturing the selected optical lens based on whether materials and mechanisms used to manufacture the selected optical lens are available.

9. The method according to claim 6, further comprising determining, by the processing circuitry, out of a plurality of possible apparatuses, an apparatus that is to manufacture the selected optical lens based on workload of each of the plurality of possible apparatuses.

10. The method according to claim 6, wherein the determining of the possible modification to the first optical lens or the second optical lens that is both compatible with the prescription information and associated with the additional information, is based on a trend among a particular age group a user belongs to, a hobby of the user, and a current time of the year.

11. A non-transitory computer-readable storage medium including computer executable instructions, wherein the instructions, when executed by a computer, cause the computer to perform a method, the method comprising:
receiving, via a network, lens data;
receiving, via a network from a client device, prescription information that includes one or more of distance, addition, sphere, cylinder, axis, and prism;
determining a first optical lens based on the prescription information received from the client device;
determining additional information to request based at least on the determined first optical lens and lens data, the additional information including one of lens-specific data and non-lens-specific data;
transmitting, via the network to the client device, a result of the initial search along with a request for the additional information;
in response to receiving the additional information via the network from the client device, determining at least one of a possible modification to the first optical lens and a second optical lens based on the first determined optical lens and the additional information;
transmitting, via the network to the client device, an updated result that includes the first optical lens as well as the possible modification to the first optical lens or the second optical lens; and
receiving, via the network from the client device, a selection of an optical lens from the updated result out of one of the first optical lens, the first optical lens with the possible modification, and the second optical lens.

12. The non-transitory computer-readable storage medium according to claim 11, wherein the lens-specific data includes one of pantoscopic tilt and a corridor length, and the non-lens-specific data includes one of age, a medical condition, and a geographical location of a user who is associated with the prescription information.

13. The non-transitory computer-readable storage medium according to claim 11, further comprising determining a timeline for manufacturing the selected optical lens based on whether materials and mechanisms used to manufacture the selected optical lens are available.

14. The non-transitory computer-readable storage medium according to claim 11, further comprising determining out of a plurality of possible apparatuses, an apparatus that is to manufacture the selected optical lens based on workload of each of the plurality of possible apparatuses.

15. The non-transitory computer-readable storage medium according to claim 11, wherein the determining of the possible modification to the first optical lens or the second optical lens that is both compatible with the prescription information and associated with the additional information, is based on a trend among a particular age group a user belongs to, a hobby of the user, and a current time of the year.
